(19) United States
(12) Patent Application Publication
(10) Pub. No.: US 2025/0279209 A1
(43) Pub. Date: Sep. 4, 2025

(54) PROVISION OF A MEDICAL RISK ASSESSMENT FOR NONCLASSICAL 21-HYDROXYLASE DEFICIENCY

(71) Applicants: SORBONNE UNIVERSITE, PARIS (FR); INSTITUT NATIONAL DE LA SANTE ET DE LA RECHERCHE MEDICALE (INSERM), PARIS (FR); ASSISTANCE PUBLIQUE - HOPITAUX DE PARIS, Paris 12 (FR); CENTRE NATIONAL DE LA RECHERCHE SCIENTIFIQUE, PARIS (FR); MUSEUM NATIONAL D'HISTOIRE NATURELLE, PARIS (FR); UNIVERSITE PARIS-SACLAY, GIF-SUR-YVETTE (FR)

(72) Inventors: Antonin LAMAZIERE, PARIS (FR); Guillaume BACHELOT, PARIS (FR); Jacques YOUNG, LE KREMLIN-BICÊTRE (FR); Anne BACHELOT, PARIS (FR); Yves LEBOUC, PARIS (FR)

(21) Appl. No.: 18/858,423

(22) PCT Filed: Apr. 21, 2022
(86) PCT No.: PCT/FR2022/050759

§ 371 Date: Oct. 21, 2024

(51) Int. Cl. G16H 50/30; G16H 10/40; G16H 10/60; G16H 50/70

(52) U.S. Cl. CPC G16H 50/30; G16H 10/40; G16H 10/60; G16H 50/70

(57) ABSTRACT

The disclosure relates to an automated system for processing physiological parameter measurements for providing a medical risk assessment, wherein it comprises: a module for receiving physiological parameter measurements (p1 . . . p15) for a female to be assessed; and a module for calculating a nonclassical 21-hydroxylase deficiency score (SNC21OHD) for the female to be assessed, according to a model which gives the score (SNC21OHD) on the basis of the measurements (p1 . . . p15) received. The physiological parameters comprise at least the following steroids: 21-deoxycortisol; 21-deoxycorticosterone; 11β-hydroxyandrostenedione; 17-OH-progesterone; androstenedione; corticosterone; and testosterone.

### CROSS-REFERENCE TO RELATED APPLICATION(S)

This present application is a national stage application of International Patent Application No. PCT/FR2022/050759, filed Apr. 21, 2022, the disclosure of which is hereby incorporated by reference in its entirety.

### TECHNICAL FIELD

The present disclosure falls within the field of Nonclassical 21-hydroxylase deficiency (NC21OHD). More particularly, the present disclosure relates to systems, methods and computer programs for providing an assessment of a medical risk of nonclassical 21-hydroxylase deficiency (NC21OHD), and a method for assessing a medical risk of nonclassical 21-hydroxylase deficiency (NC21OHD).

### BACKGROUND

The aim of the article “Androgen excess and diagnostic steroid biomarkers for nonclassical 21-hydroxylase deficiency without cosyntropin stimulation” by TURCU et al. published in the European Journal of Endocrinology, 183 63-71, in 2020, was to identify steroids that would allow reliable diagnosis of nonclassical 21-hydroxylase deficiency (NC21OHD). To this end, different steroids were measured for each female in a cohort and, using logistic regression modelling, three steroids were identified as being particularly discriminating in combination: 21-deoxycortisol, 17-OH-progesterone and corticosterone.

The article stresses that the measurements must be taken at the beginning of the follicular phase of the menstrual cycle, which is difficult to implement in some cases.

In addition, the model based on the combination of the three steroids mentioned above has not been validated on an independent cohort, so the robustness of this model may be questionable.

### SUMMARY

The aim of the disclosure is to overcome some or all of these problems.

To this end, an automated system is proposed for processing physiological parameters measurements for providing a medical risk assessment, wherein it comprises:
a module for receiving physiological parameters measurements for a female to be assessed; and
a module for calculating a nonclassical 21-hydroxylase deficiency score for the female to be assessed, according to a model giving the score from the measurements received; the physiological parameters comprising at least the following steroids:
21-deoxycortisol,
21-deoxycorticosterone;
11β-hydroxyandrostenedione;
17-OH-progesterone;
androstenedione;
corticosterone; and
testosterone.

Surprisingly, the use of the above steroids allows a reliable and robust assessment of nonclassical 21-hydroxylase deficiency (NC21OHD), even when measurements are taken during the luteal phase of the menstrual cycle. In this way, the steroids selected mean that we are no longer dependent on the time at which the measurements are taken.

Optionally, the physiological parameters also comprise at least one of the following steroids:
pregnenolone;
dehydroepiandrosterone;
cortisol;
cortisone;
11-deoxycorticosterone;
17-OH-pregnenolone;
aldosterone; and
progesterone.

Optionally, the physiological parameters may also comprise steroids only.

Optionally, the model giving the nonclassical 21-hydroxylase deficiency score is previously trained by machine learning.

Optionally, the model giving the nonclassical 21-hydroxylase deficiency score comprises a linear combination of the measurements of the physiological parameters.

Optionally, at least one of the steroids was measured during a luteal phase of a menstrual cycle of the female to be assessed.

Optionally, the automated system also comprises:
a module for comparing the nonclassical 21-hydroxylase deficiency score with a predefined threshold, for example 0.5; and
a module for calculating, when the nonclassical 21-hydroxylase deficiency score is on a predefined side of the threshold, a polycystic ovary syndrome score of the female to be assessed, according to a model giving the polycystic ovary syndrome score from at least some of the measurements received.

Optionally, the polycystic ovary syndrome score may also be obtained from at least the steroid measurements: 11β-hydroxyandrostenedione and androstenedione, preferably from measurements of the steroids: testosterone and corticosterone.

A method for obtaining a model for an automated system according to the disclosure is also proposed, comprising:
for each of several female in a cohort, obtaining measurements of the physiological parameters of a female and a status indicating that this female either has the nonclassical 21-hydroxylase deficiency or does not have this deficiency; and
determining the model, by training the model using a training algorithm on the basis of measurements of the physiological parameters of the female in the cohort.

Optionally, all the steroids were measured during a follicular phase of a menstrual cycle.

Also proposed is a method for automated processing of physiological parameters measurements in order to provide an assessment of a medical risk, wherein it comprises:
receiving measurements of physiological parameters of a female to be assessed; and
using a model to provide a nonclassical 21-hydroxylase deficiency score for the female to be assessed, based on the measurements received;
   the physiological parameters comprising at least the following steroids:
21-deoxycortisol;
21-deoxycorticosterone;
11β-hydroxyandrostenedione;
17-OH-progesterone;
androstenedione;
corticosterone; and
testosterone.

Optionally, the physiological parameters further comprise at least one of the following steroids:
pregnenolone;
dehydroepiandrosterone;
cortisol;
cortisone;
11-deoxycorticosterone;
17-OH-pregnenolone;
aldosterone; and
progesterone.

Optionally, the physiological parameters comprise steroids only.

Optionally, the model giving the nonclassical 21-hydroxylase deficiency score is previously trained by machine learning.

Optionally, the model giving the nonclassical 21-hydroxylase deficiency score comprises a linear combination of the measurements of the physiological parameters.

Optionally, at least one of the steroids was measured during a luteal phase of a menstrual cycle of the female to be assessed.

Optionally, the method also comprises:
comparing the nonclassical 21-hydroxylase deficiency score with a predefined threshold, for example 0.5; and
calculating, when the nonclassical 21-hydroxylase deficiency score is on a predefined side of the threshold, a polycystic ovary syndrome score of the female to be assessed, according to a model giving the score of polycystic ovary syndrome from at least one part of the measurements received.

Optionally, the polycystic ovary syndrome score may also be obtained from at least the measurements of the steroid: 11β-hydroxyandrostenedione and androstenedione, preferably also from measurements of the steroids: testosterone and corticosterone.

Also proposed is a computer program that may be downloaded from a communication network and/or recorded on a computer-readable medium, wherein it comprises instructions for executing a method according to the disclosure, when the computer program is executed on a computer.

Also proposed is a method for assessing a medical risk of a nonclassical 21-hydroxylase deficiency in a female to be assessed, comprising:
collecting physiological parameters from the female to be assessed, the physiological parameters comprising at least the following steroids: 21-deoxycortisol, 21-deoxycorticosterone, 11β-hydroxyandrostenedione, 17-OH-progesterone, androstenedione, corticosterone and testosterone;
calculating a nonclassical 21-hydroxylase deficiency score by introducing these physiological parameters into an automated system according to the disclosure; and
comparing this score with a threshold, the female to be assessed being considered to be at risk of nonclassical 21-hydroxylase deficiency if the score is below the threshold.

Optionally, at least one of the steroids is measured during a luteal phase of a menstrual cycle of the female to be assessed.

### BRIEF DESCRIPTION OF THE DRAWINGS

The disclosure will be better understood with the aid of the following description, given only by way of example and made with reference to the attached drawings wherein:

FIG. 1 is a simplified view of an automated data processing system according to the disclosure for providing an assessment of a medical risk, in particular of nonclassical 21-hydroxylase deficiency (NC21OHD), on the basis of physiological parameters of a female to be assessed,

FIG. 2 is a block diagram illustrating the steps of a method according to the disclosure for processing data to provide an assessment of a medical risk, in particular of nonclassical 21-hydroxylase deficiency (NC21OHD),

FIG. 3 groups together several views of the system shown in FIG. 1, displaying information relating to the assessment carried out, in several different scenarios,

FIG. 4 is a block diagram illustrating the steps in a method for obtaining an M_{NC2IOHD} model for assessing a medical risk of nonclassical 21-hydroxylase deficiency (NC21OHD) and an M_{PCOS} model for assessing a medical risk of polycystic ovary syndrome (PCOS),

FIG. 5 is a score plot resulting from an Orthogonal Partial Least Squares-Discriminant Analysis (OPLS-DA) carried out on a main development cohort to obtain the M_{NC2IOHD} model of the system shown in FIG. 1,

FIG. 6 is a score plot after using the M_{NC2IOHD} model on a validation cohort,

FIG. 7 repeats the score plot from FIG. 6, without the measurements from the validation cohort of female with a polycystic ovary syndrome (PCOS), and distinguishing between the measurements from control female and the measurements from female with nonclassical 21-hydroxylase deficiency (NC21OHD),

FIG. 8 repeats the score plot from FIG. 7, with the measurements from the validation cohort of female with polycystic ovary syndrome (PCOS),

FIG. 9 repeats the score plot from FIG. 6, isolating the female for whom at least one physiological parameter was measured in the luteal phase of their menstrual cycle,

FIG. 10 is a Variable Importance in Projection (VI P) plot of steroids used in the M_{NC2IOHD} model,

FIG. 11 is a Variable Importance in Projection plot of the steroids used by the M_{PCOS} model, and

FIG. 12 is a score plot resulting from a Partial Least Squares Discriminant Analysis (OPLS-DA) carried out on a development cohort referred to as annex, to obtain the M_{PCOS} model.

### DETAILED DESCRIPTION

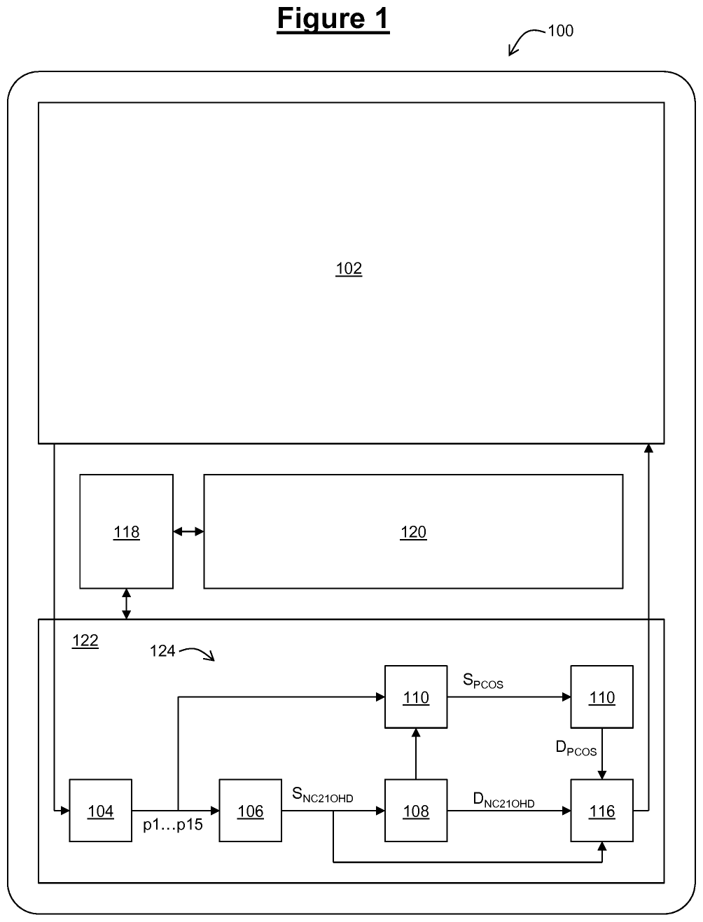

With reference to FIG. 1, an example of an automated data processing system 100 according to the disclosure will now be described. More specifically, as will become apparent later, the system 100 is designed to provide an assessment of a medical risk that a female has nonclassical 21-hydroxylase deficiency (NC21OHD). This assessment is provided, for example, in the form of a S_{NC2IOHD} score for nonclassical 21-hydroxylase deficiency (NC21OHD). In this way, the system 100 provides clinical decision support, for example for the practitioner.

The system 100 firstly comprises a man/machine interface 102 comprising at least one information input device and at least one information presentation device, for example a visual presentation device such as a screen. For example, the man/machine interface 102 comprises a touch screen acting as both an information input device and an information presentation device. For example, the system 100 is a tablet computer or a smartphone.

The system 100 also comprises a data reception module 104, designed to receive physiological parameters measurements of the female to be assessed, the physiological parameters comprise at least the following steroids: 21-deoxycortisol (21 DF), 21-deoxycorticosterone (21DB), 11β-hydroxyandrostenedione (11bOHA), 17-OH-progesterone (17OHP), androstenedione (A), corticosterone (B), and testosterone (T). Preferably, the physiological parameters also comprise at least one of the following steroids: pregnenolone (Preg), dehydroepiandrosterone (DHEA), cortisol (F), cortisone (E), 11-deoxycorticosterone (DOC), 17-OH-pregnenolone (17-OHPreg), aldosterone (ALDO), and progesterone (P). Even more preferably, the physiological parameters comprise only steroids.

In the example shown in FIG. 1, the measurements of the fifteen steroids listed above are received by the data reception module 104, and noted p1 . . . p15.

For example, the data reception module 104 is designed to cooperate with the man/machine interface 102 to receive the measurements p1 . . . p15. For example, a user of the system 100 may enter the measurements p1 . . . p15 by hand via the man/machine interface 102. Alternatively, all or some of the measurements p1 . . . p15 may be received from a remote device (not shown), such as a computer, via a wireless or wired connection.

The physiological parameters form a multidimensional space (of dimension fifteen when all the steroids indicated above are used) of which each set of measurements p1 . . . p15 is a point.

The system 100 also comprises a module 106 for calculating the S_{NC2IOHD} score of the female to be assessed. This calculation module 106 is designed to calculate the S_{NC2IOHD} score according to a M_{NC2IOHD} model, for example previously trained by machine learning.

For example, the M_{NC2IOHD} model is a linear combination, so the S_{NC2IOHD} score is a linear combination of the received measurements p1 . . . p15. In this linear combination, the measurements p1 . . . p15 are assigned with weights w1 . . . w15 respectively, and the weighted measurements p1 . . . p15 are summed to obtain the S_{NC2IOHD} score. In this case, the S_{NC2IOHD} score is given by the following formula:

$${S}_{NC 21 OHD} = {\sum }_{n = 1}^{N} wn \cdot pn + ε \qquad [ Math . 1 ]$$

where N is the number of physiological parameters measured (for example fifteen in the example in FIG. 1), and s is a constant representing a residual. This constant may be zero.

As will be explained in more detail later, the machine learning comprises determining the weights w1 . . . w15 allowing the S_{NC2IOHD} score to be representative of the risk of nonclassical 21-hydroxylase deficiency (NC21OHD) in the female to be assessed.

The machine learning may be carried out outside the automated system 100. In this case, the data reception module 104 may also be designed to receive an update containing the weights w1 . . . w15. This update is received, for example, from a remote device (not shown), such as a computer, via a wireless or wired connection.

Alternatively, the M_{NC2IOHD} model could comprise a neural network.

Generally speaking, the M_{NC2IOHD} model implements a function including parameters and the prior machine learning of the M_{NC2IOHD} model comprises determining these parameters, allowing the S_{NC2IOHD} score to be representative of the risk of nonclassical 21-hydroxylase deficiency (NC21OHD).

Preferably, the M_{NC2IOHD} model is pre-trained so that the S_{NC2IOHD} score lies within a predefined range, for example between 0 and 1.

In addition, the system 100 comprises, for example, a module 108 for comparing the S_{NC2IOHD} score with a predefined threshold T_{NC2IOHD}, for example 0.5. When the S_{NC2IOHD} score is on a predefined side (above or well below) the T_{NC2IOHD} threshold, the female to be assessed is considered to be at high risk of nonclassical 21-hydroxylase deficiency (NC21OHD), and at low risk on the other side. The result of this comparison provides a D_{NC2IOHD} assessment of the risk of nonclassical deformed 21-hydroxylase deficiency (NC21OHD). For example, the D_{NC2IOHD} risk assessment is binary and indicates either a high risk of nonclassical deformed 21-hydroxylase deficiency (NC21OHD) when the S_{NC2IOHD} score is above the T_{NC2IOHD} threshold, or a low risk of nonclassical deformed 21-hydroxylase deficiency (NC21OHD) when the S_{NC2IOHD} score is below the T_{NC2IOHD} threshold, or vice versa. Alternatively, the D_{NC2IOHD} risk assessment could be more gradual, for example additionally indicating a very low risk when the S_{NC2IOHD} score is well below the T_{NC2IOHD} threshold (e.g. below 0.25) and/or a very high risk when the S_{NC2IOHD} score is well above the T_{NC2IOHD} threshold (e.g. above 0.75).

The system 100 also comprises a module 110 for calculating, when the S_{NC2IOHD} score is on the predefined side of the T_{NC2IOHD} threshold indicating a low risk of nonclassical 21-hydroxylase deficiency (NC21OHD), a S_{pcos} polycystic ovary syndrome (PCOS) score for the female to be assessed, on the basis of at least some of the measurements p1 . . . p15, for example all of them, or even just all of these measurements. This calculation module 110 is designed to calculate the S_{pcos} score according to an M_{PCOS} model, for example previously trained by machine learning.

For example, the M_{PCOS} model is a linear combination, so the S_{pcos} score is a linear combination of the p1 . . . p15 measurements used. In this linear combination, the measurements p1 . . . p15 used are respectively assigned weights w′1 . . . w′15, and the weighted measurements p1 . . . p15 are summed to obtain the S_{pcos} score. In this case, the S_{NC2IOHD} score is given by the following formula:

$${S}_{PCOS} = {\sum }_{n = 1}^{N} {w}^{′} n \cdot pn + {ε}^{′} \qquad [ Math . 2 ]$$

where N is the number of measured physiological parameters used (e.g. fifteen in the example in FIG. 1), and s′ is a constant representing a residual. This constant may be zero.

As will be explained in more detail later, the machine learning comprises determining the weights w′1 . . . w′15 allowing the S_{PCOS} score to be representative of the risk that the female being assessed has polycystic ovary syndrome (PCOS).

The machine learning may be carried out outside the automated system 100. In this case, the data reception module 104 may also be designed to receive an update containing the weights w′1 . . . w′15. This update is received, for example, from a remote device (not shown), such as a computer, via a wireless or wired connection.

Alternatively, the M_{PCOS} model could comprise a neural network.

In general, the M_{PCOS} model implements a function including the parameters and the prior machine learning of the M_{PCOS} model comprises the determination of these parameters allowing the S_{PCOS} score to be representative of the risk of developing polycystic ovary syndrome (PCOS).

Preferably, the M_{PCOS} model is pre-trained so that the S_{PCOS} score lies within a predefined range, for example between 0 and 1.

In addition, the system 100 comprises, for example, a module 112 for comparing the S_{PCOS} score with a predefined T_{PCOS} threshold, for example 0.5. When the S_{PCOS} score is on a predefined side (above or well below) the T_{PCOS} threshold, the female to be assessed is considered to be at high risk of polycystic ovary syndrome, and at low risk on the other side. The result of this comparison provides a D_{PCOS} assessment of the risk of polycystic ovary syndrome. For example, the D_{PCOS} risk assessment is binary and indicates either a low risk of polycystic ovary syndrome (PCOS) when the S_{PCOS} score is above the T_{PCOS} threshold, or a high risk of polycystic ovary syndrome (PCOS) when the S_{PCOS} score is below the T_{PCOS} threshold, or vice versa. Alternatively, the D_{PCOS} risk assessment could be more gradual, for example by also indicating a very low risk when the S_{PCOS} score is well below the T_{PCOS} threshold (e.g. below 0.25) and/or a very high risk when the S_{PCOS} score is well above the T_{PCOS} threshold (e.g. above 0.75).

The system 100 also comprises an information presentation module 116 designed to present the S_{NC2IOHD} score and/or the D_{NC2IOHD} risk assessment to a user of the system 100. The presentation module 116 may also be designed to present the S_{pcos} score and/or the D_{PCOS} risk assessment.

The presentation module 116 is designed, for example, to cooperate with the man/machine interface 102. For example, the presentation module 116 is designed to cause information to be displayed on the man/machine interface 102, for example on the touch screen when such a screen is used.

Furthermore, the system 100 comprises, for example, a computer system comprising a processing unit 118 (such as one or more microprocessors) and a main memory 120 coupled to the processing unit 118. A computer program 124 containing computer program instructions is configured to be loaded, either in its entirety or sequentially in chunks, into the main memory 120. The loaded instructions may then be executed by the processing unit 118. For example, the system 100 also comprises a memory of mass 122 wherein the computer program 124 is stored before being loaded into the main memory 120.

For example, the modules described above are implemented in the computer program in the form of software modules.

For example, particularly where the system 100 is a smartphone, the computer program 124 may be in the form of an application (APP), for example available from the Apple (trademark) and/or Android (trademark) app shop.

Alternatively, all or some of the modules could be implemented in the form of hardware modules, i.e. in the form of an electronic circuit, for example micro-wired, not involving a computer program.

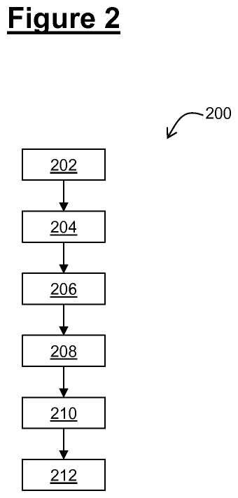

With reference to FIG. 2, an example 200 of an automated data processing method, in the context of the example system 100 of FIG. 1, will now be described.

During a step 202, the reception module 104 receives the measurements p1 . . . p15.

In a step 204, the calculation module 106 implementing the M_{NC2IOHD} model calculates the S_{NC2IOHD} score from the measurements p1 . . . p15 received.

In a step 206, the comparison module 108 compares the S_{NC2IOHD} score with the predefined threshold and determines the D_{NC2IOHD} risk assessment.

In a step 208, if the S_{NC2IOHD} score is on the side of the T_{NC2IOHD} threshold indicating a low risk of nonclassical 21-hydroxylase deficiency (NC21OHD), for example if the S_{NC2IOHD} score is less than 0.5, the calculation module 110 implementing the M_{PCOS} model calculates the Spcos score from at least some of the measurements p1 . . . p15 received.

In a step 210, the comparison module 110 compares the S_{PCOS} score with the T_{PCOS} threshold and determines the D_{PCOS} risk assessment.

During a step 212, the information presentation module 116 presents the S_{NC2IOHD} score and/or the D_{NC2IOHD} risk assessment, as well as, if applicable, the S_{PCOS} score and/or the D_{PCOS} risk assessment.

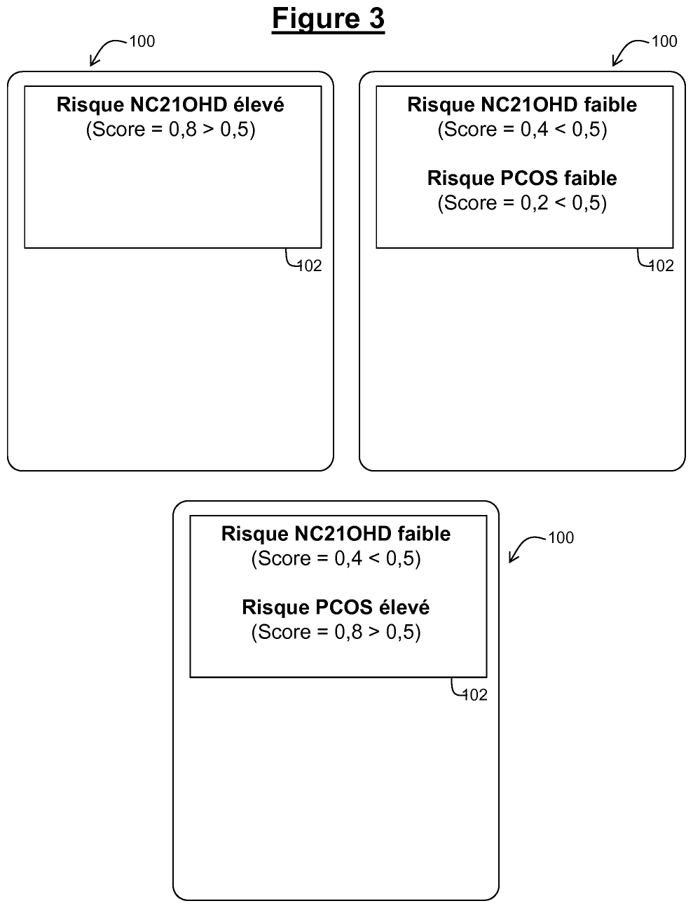

With reference to FIG. 3, during step 212, the presentation module 116 causes, for example, a sentence representing the D_{NC2IOHD} risk assessment to be displayed, for example “high NC21OHD risk” or “low NC21OHD risk”, depending on the D_{NC2IOHD} risk assessment. Still for example, the presentation module 116 causes the S_{NC2IOHD} score and, preferably, the predefined threshold to be displayed. The latter is 0.5 in the example shown in FIG. 3. In addition, when the S_{NC2IOHD} score is on the side of the T_{NC2IOHD} threshold indicating a low risk of nonclassical 21-hydroxylase deficiency (NC21OHD), the presentation module 116 causes, for example, a sentence representing the D_{PCOS} risk assessment of polycystic ovary syndrome (PCOS) to be displayed, for example “high PCOS risk” or “low PCOS risk”, depending on the D_{PCOS} risk assessment. Still for example, the presentation module 116 causes the S_{PCOS} score and, preferably, the T_{PCOS} threshold to be displayed.

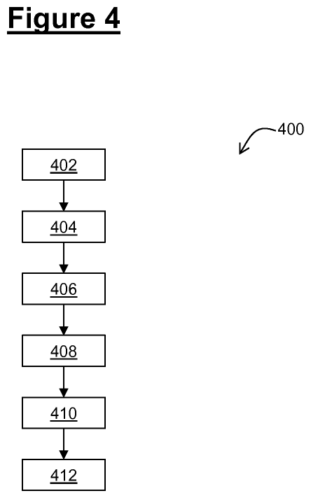

With reference to FIG. 4, a method 400 for obtaining the M_{NC2IOHD} and M_{PCOS} models for an automated system such as that shown in FIG. 1 will now be described.

In a step 402, for each of several female in a so-called main development cohort, measurements p1 . . . p15 of physiological parameters of the woman are obtained, as well as a status of nonclassical 21-hydroxylase deficiency (NC21OHD) of this female. For example, for all the female in the main development cohort, measurements are taken during the follicular phase of the menstrual cycle. The physiological parameters comprise at least the following steroids: 21-deoxycortisol (21 DF), 21-deoxycorticosterone (21DB), 11β-hydroxyandrostenedione (11bOHA), 17-OH-progesterone (17OHP), androstenedione (A), corticosterone (B), and testosterone (T). Preferably, the physiological parameters also comprise at least one of the following steroids: pregnenolone (Preg), dehydroepiandrosterone (DHEA), cortisol (F), cortisone (E), 11-deoxycorticosterone (DOC), 17-OH-pregnenolone (17-OHPreg), aldosterone (ALDO), and progesterone (P). Even more preferably, the physiological parameters comprise only steroids.

The status is binary and indicates either a nonclassical 21-hydroxylase deficiency (NC21OHD) or the absence of this deficiency. For the method 400 to function correctly, the status of at least one female indicates that this female has a nonclassical 21-hydroxylase deficiency (NC21OHD) and the status of at least one other female indicates the absence of this deficiency.

For some of the female in the main development cohort, the measurements for certain steroids may be missing.

In addition, in step 402, measurements p1 . . . p15 of physiological parameters from each of several female in a validation cohort are obtained, together with a nonclassical 21-hydroxylase deficiency (NC21OHD) status for that female. Preferably, polycystic ovary syndrome (PCOS) status is also obtained for each female in the validation cohort.

In a step 404, the measurements from the main development cohort are analyzed to look for statistically outlier measurements for one or more female in the main development cohort. In addition, each female whose measurements were statistically aberrant was removed from the main development cohort. To carry out this data analysis, for example, the measurements are first centered and reduced. Principal Component Analysis (PCA) may then be used, for example, to search for one or more sets of statistically outlier measurements.

In a step 406, the M_{NC2IOHD} model is built by machine learning from measurements of physiological parameters for the female in the main development cohort. The aim of machine learning is for the M_{NC2IOHD} model to provide a S_{NC2IOHD} score that reliably predicts the status of female in the main development cohort and, subsequently, of a new female whose measurements of steroids are provided to the M_{NC2IOHD} model. A training algorithm is generally used for this. This is designed to adjust parameters of the M_{NC2IOHD} model from training data comprising input data and output data, so that the M_{NC2IOHD} model provides, from known input data, the known output data.

For example, the M_{NC2IOHD} model is a linear combination of measurements of physiological parameters and machine learning is designed to determine weights of this linear combination, so that the linear combination reliably predicts the statuses of female in the main development cohort.

For example, an Orthogonal Partial Least Squares-Discriminant Analysis (OPLS-DA) is used.

Taking steroid measurements on female is a difficult operation to implement, so measurements may generally only be obtained for a small number of female, for example a few dozen to a few hundred. In addition, some measurements may be missing. In addition, it is reasonable to assume that the steroids are a priori multi-collinear. The OPLS-DA analysis therefore seems particularly appropriate as it is effective in the case of multicollinearity, even in a small cohort. It is also robust, meaning that it may be carried out even with some measurements missing.

As is well known, by noting X a matrix grouping the measurements of physiological parameters for the female in the main development cohort and Y a vector grouping the status of these females, the OPLS-DA analysis decomposes the X and Y matrices as follows:

$$X = {TP}^{T} + {E}_{X} \qquad [ Math . 3 ]$$

$$Y = {UQ}^{T} + {E}_{Y}$$

where T and U are the scores of the X and Y matrices respectively, P and Q are the loadings of the X and Y matrices respectively, and Ex and E_{Y} are the residuals, which the decomposition seeks to make as small as possible.

The weights of the linear combination, noted wn with n=1 . . . N where N is the number of physiological parameters, are then the components of a vector W defined as follows:

$$T = {XW}^{*} \qquad [ Math . 4 ]$$

W* being the trans-conjugate vector of the vector W.

The M_{NC2IOHD} model is therefore the linear combination of the measurements of the physiological parameters, with the weights corresponding respectively to the components wn of the vector W.

In a step 408, the M_{NC2IOHD} model may be evaluated in order to validate or invalidate it.

The evaluation may be an internal evaluation (i.e. based on the main development cohort) and/or an external evaluation (i.e. based on the validation cohort).

For example, the internal evaluation may use one or more of the following: the coefficient of determination R², the permutation test, the prediction coefficient Q² and the average accuracy (i.e. the proportion of correct predictions made by the M_{NC2IOHD} model).

For example, the external evaluation uses the receiver operating characteristic (ROC) of the validation cohort. For example, an Area Under the Curve (AUC) of the ROC curve may be compared with a predefined threshold. The average accuracy may also be used for the external evaluation, based on the validation cohort (validation accuracy).

In step 410, the M_{PCOS} model is built by machine learning from measurements of the physiological parameters p1 . . . p15 for female in a so-called annex development cohort. This annex development cohort is made up, for example, of control female from the validation cohort and female with polycystic ovary syndrome (PCOS). The aim of machine learning is for the M_{PCOS} model to provide a S_{PCOS} score that reliably predicts the status of female in the annex development cohort and, subsequently, of a new female whose steroid measurements are provided to the M_{PCOS} model. A training algorithm is generally used for this. The latter is designed to adjust parameters of the M_{PCOS} model from training data comprising input data and output data, so that the M_{PCOS} model provides, from known input data, the known output data.

For example, the M_{PCOS} model is a linear combination of measurements of physiological parameters and machine learning is designed to determine weights of this linear combination, so that the linear combination reliably allows to predict the statuses of female in the annex development cohort.

For example, an Orthogonal Partial Least Squares-Discriminant Analysis (OPLS-DA) is used.

In a step 412, the M_{PCOS} model may be evaluated in order to validate or invalidate it. For example, a cross-validation based on the annex development cohort may be carried out at the same time as the machine learning.

An example of implementation of the method 400 may be used is described below.

### Stage 402: Obtaining the Measurements

In the example described, the main development cohort comprises 38 females, including 19 with nonclassical 21-hydroxylase deficiency (NC21OHD) and 19 so-called control female, i.e. with neither this deficiency nor polycystic ovary syndrome (PCOS). For its part, the validation cohort comprises, in the example described, 355 females, including 17 with nonclassical 21-hydroxylase deficiency (NC21OHD), 266 females with polycystic ovary syndrome (PCOS) and 72 so-called control females, i.e. female with none of these diseases.

In addition, in the example described, the fifteen steroids detailed above were measured in each female in the main development cohort, during the follicular phase of their menstrual cycle. These same fifteen steroids were measured in every female in the validation cohort, during the luteal phase of their menstrual cycle for some of the female and during the follicular phase for the others.

### Step 404: Statistical Verification of the Measurements

In the example described, no statistically outlier measurements were found in the measurements of the main development cohort.

### Step 406: Obtaining the M_{NC2IOHD} Model

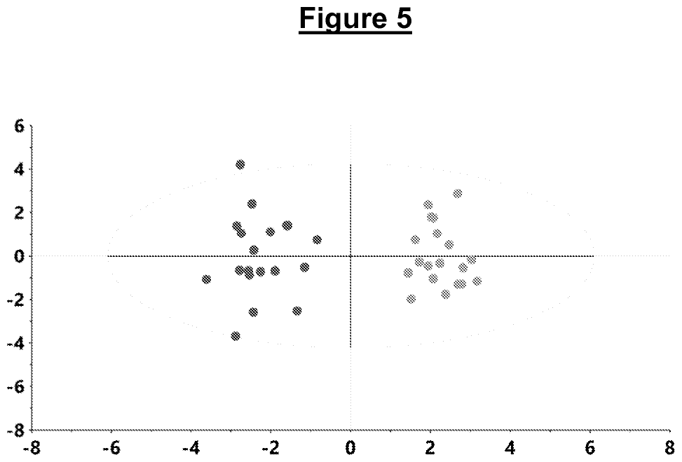

The main development cohort is used to determine the M_{NC2IOHD} model from the measurements of the fifteen steroids. In the example described, the OPLS-DA analysis is used, so that the M_{NC2IOHD} model obtained is a linear combination of the measurements of the fifteen steroids. FIG. 5 is a score plot of the measurements of the females in the main development cohort. As may be seen, the measurements of the females with nonclassical 21-hydroxylase deficiency (NC21OHD) (dark grey dots) and the measurements of the control females (light grey dots) form two distinct groups, to the left and right of the central vertical line respectively.

### Step 408: Validation of the M_{NC2IOHD}7 Model

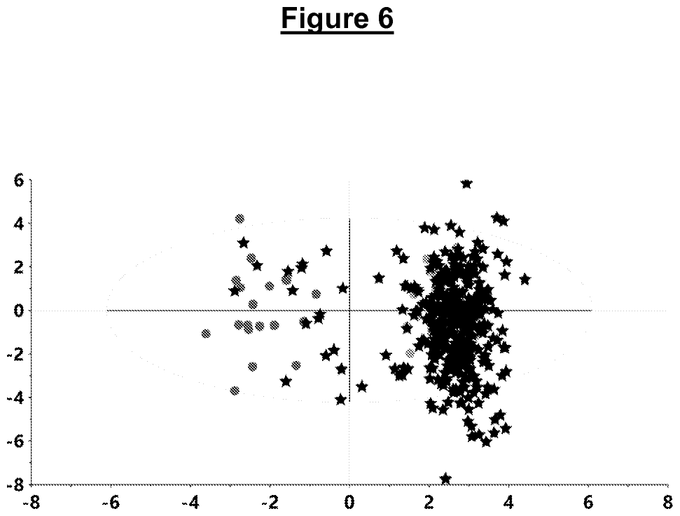

FIG. 6 is a score plot of the measurements of the females in the main development cohort of (dark grey dots and light grey dots, as in FIG. 5) and the measurements of the females in the validation cohort (black stars), with no distinction for the latter between control females, the females with nonclassical 21-hydroxylase deficiency (NC21OHD) and the females with polycystic ovary syndrome (PCOS).

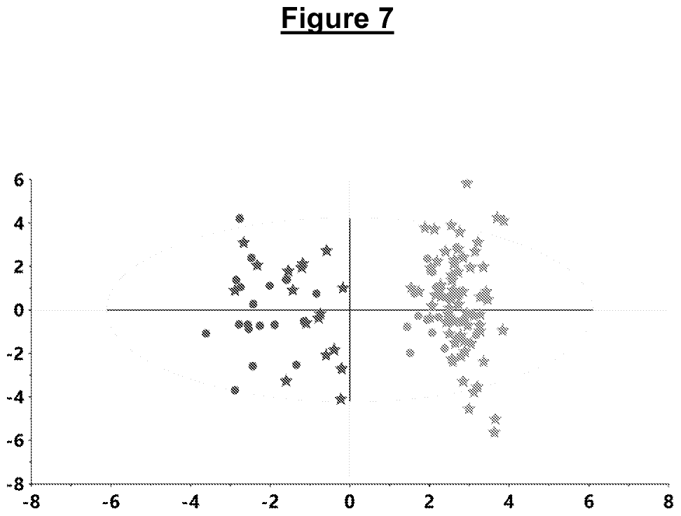

FIG. 7 is similar to FIG. 6, without the females with polycystic ovary syndrome (PCOS) and distinguishing the control females (light grey stars) from the females with nonclassical 21-hydroxylase deficiency (NC21OHD) (dark grey stars).

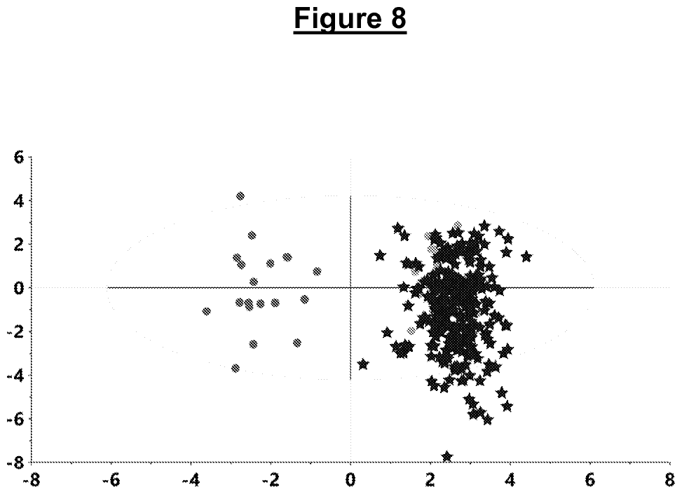

FIG. 8 is similar to FIG. 7, without the control females and the females with nonclassical 21-hydroxylase deficiency (NC21OHD), but with the females with polycystic ovary syndrome (PCOS) (black stars).

As may be seen in FIG. 6, FIG. 7 and FIG. 8, the M_{NC2IOHD} model obtained allow the females with nonclassical 21-hydroxylase deficiency (NC21OHD) to be distinguished not only from control females, but also from females with polycystic ovary syndrome (PCOS).

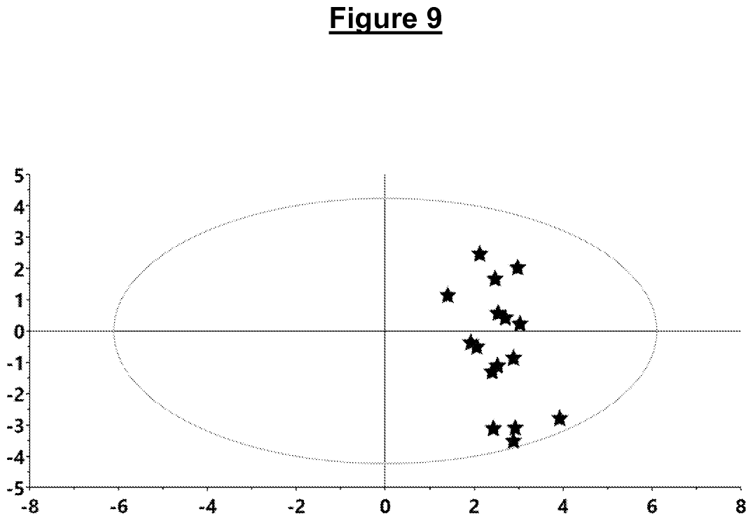

FIG. 9 is a score plot of the measurements of the control female and having the polycystic ovary syndrome (PCOS) in the validation cohort, for whom steroids were measured during the luteal phase of their menstrual cycle. As may be seen, despite the measurements in the luteal phase, they are well recognized as being at low risk of nonclassical 21-hydroxylase deficiency (NC21OHD).

Surprisingly, a reliable assessment was obtained even for the females whose measurements were taken during the luteal phase of their menstrual cycle. In this way, the steroids selected mean that we are no longer dependent on the time at which the measurements are taken.

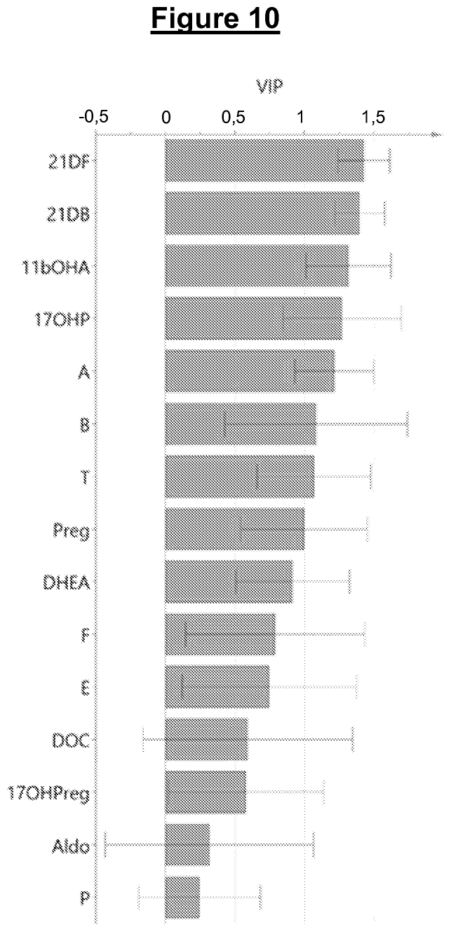

FIG. 10 is a Variable Importance in Projection (VIP) plot of the fifteen steroids in the M_{NC2IOHD} model.

As may be seen from this FIG., the seven most important steroids are: 21-deoxycortisol (21DF), 21-deoxycorticosterone (21DB), 11β-hydroxyandrostenedione (11bOHA), 17-OH-progesterone (17-OHP), androstenedione (A), corticosterone (B), and testosterone (T). It was even found that using of these seven steroids, without the eight others listed above, gave a fairly reliable assessment, even with measurements taken in the luteal phase.

It will be appreciated that new females may be added to the main development cohort to refine the M_{NC2IOHD} model. Thus, a new OPLS-DA analysis (as implemented for example in the steps of the method 400 detailed above) may be carried out with the development cohort thus enriched with new females, in order to obtain updated weights for the M_{NC2IOHD} model. These updated weights may then be transmitted to the systems 100 for updating.

### Step 410: Obtaining the M_{PCOS} Model

The annex development cohort consists of 266 females with a polycystic ovary syndrome (PCOS) and 72 control females from the validation cohort. This annex development cohort is used to obtain the M_{PCOS} model.

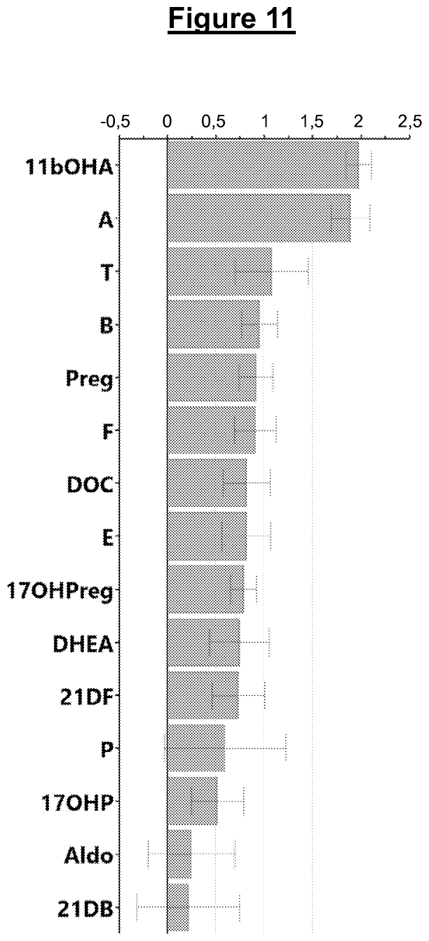

FIG. 11 is a Variable Importance in Projection (VIP) plot of the fifteen steroids in the M_{PCOS} model.

As may be seen in this FIG., the two most important steroids are androstenedione (A) and 11β-hydroxyandrostenedione (11bOHA), possibly supplemented by pregnenolone (Preg) and 21-deoxycorticosterone (21DB).

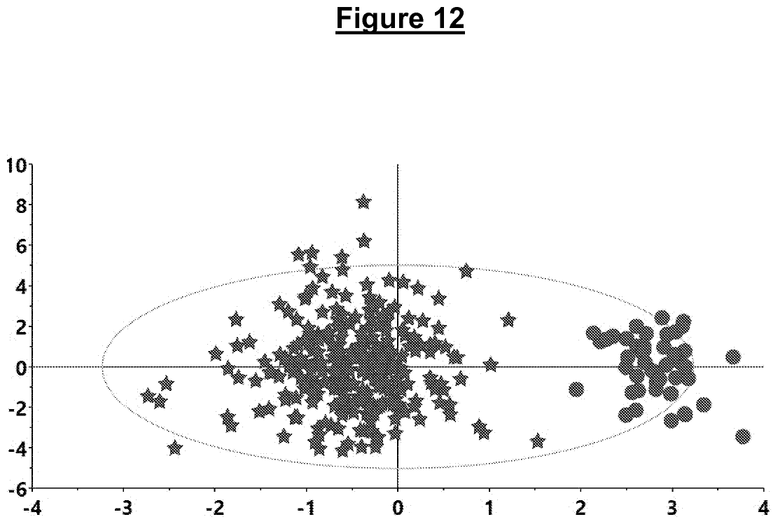

As may be seen in FIG. 12, the M_{PCOS} model obtained allow to distinguish the females with polycystic ovary syndrome (PCOS) (circles) from control females (stars).

In conclusion, it is clear that a system such as the one described above provides a reliable assessment of the risk of nonclassical 21-hydroxylase deficiency (NC21OHD).

It should be noted that the disclosure is not limited to the embodiments described above. In fact, it will appear to the person skilled in the art that various modifications may be made to the above-described embodiments, in the light of the teaching just disclosed.

In particular, the M_{PCOS} model could be used independently of the M_{NC2IOHD} model to provide an assessment of a medical risk of polycystic ovary syndrome (PCOD), for example in cases where a medical risk of nonclassical 21-hydroxylase deficiency (NC21OHD) in the female to be assessed has been ruled out by a method other than the use of the M_{NC2IOHD} model.

In the detailed presentation of the disclosure given above, the terms used should not be interpreted as limiting the disclosure to the embodiments set out in this description, but should be interpreted to comprise all equivalents the anticipation of which is within the grasp of the person skilled in the art by applying his general knowledge to the implementation of the teaching just disclosed.

1. An automated system for processing physiological parameters measurements for providing a medical risk assessment, wherein it comprises:
a module for receiving physiological parameters measurements (p1 . . . p15) for a female to be assessed; and
a module for calculating a nonclassical 21-hydroxylase deficiency score (S_{NC2IOHD}) for the female to be assessed, according to a model giving the nonclassical 21-hydroxylase deficiency score (S_{NC2IOHD}) from the measurements (p1 . . . p15) received;
the physiological parameters comprising at least the following steroids:
21-deoxycortisol (21DF);
21-deoxycorticosterone (21DB);
11β-hydroxyandrostenedione (11bOHA);
17-OH-progesterone (17OHP);
androstenedione (A);
corticosterone (B); and
testosterone (T).

2. The automated system of claim 1, wherein the physiological parameters further comprise at least one of the following steroids:
pregnenolone (Preg);
dehydroepiandrosterone (DHEA);
cortisol (F);
cortisone (E);
11-deoxycorticosterone (DOC);
17-OH-pregnenolone (170HPreg);
aldosterone (ALDO); and
progesterone (P).

3. The automated system according to claim 1, wherein the physiological parameters comprise steroids only.

4. The automated system according to claim 1, wherein the model giving the nonclassical 21-hydroxylase deficiency score (S_{NC2IOHD}) is previously trained by machine learning.

5. The automated system according to claim 4, wherein the model giving the nonclassical 21-hydroxylase deficiency score (S_{NC2IOHD}) comprises a linear combination of the measurements of the physiological parameters.

6. The automated system according to claim 1, wherein at least one of the steroids was measured during a luteal phase of a menstrual cycle of the female to be assessed.

7. The automated system according to claim 1, further comprising:
a module for comparing the nonclassical 21-hydroxylase deficiency score (S_{NC2IOHD}) with a predefined threshold, for example 0.5; and
a module for calculating, when the nonclassical 21-hydroxylase deficiency score (S_{NC2IOHD}) is on a predefined side of the threshold, a polycystic ovary syndrome score (S_{PCOS}) of the female to be assessed, according to a model giving the polycystic ovary syndrome score (S_{PCOS}) from at least some of the measurements (p1 . . . p15) received.

8. The automated system according to claim 7, wherein the score (S_{PCOS}) of polycystic ovary syndrome is given from at least the steroid measurements: 11β-hydroxyandrostenedione (11bOHA) and androstenedione (A), preferably also from measurements of the steroids: testosterone (T) and corticosterone (B).

9. A method for obtaining a model for an automated system according to claim 1, comprising:
for each of several female in a cohort, obtaining measurements of the physiological parameters of a female and a status indicating that the female either has the nonclassical 21-hydroxylase deficiency or does not have this deficiency; and
determining the model, by training the model using a training algorithm, on the basis of measurements of the physiological parameters of the female in the cohort.

10. The method of claim 9, wherein all steroids were measured during a follicular phase of a menstrual cycle.

11. The method for automated processing of physiological parameters measurements in order to provide an assessment of a medical risk, comprising:
receiving measurements (p1 . . . p15) of physiological parameters of a female to be assessed; and
using a model to provide a nonclassical 21-hydroxylase deficiency score (S) for the female to be assessed, based on the measurements (p1 . . . p15) received;
the physiological parameters comprising at least the following steroids:
21-deoxycortisol (21DF);
21-deoxycorticosterone (21DB);
11β-hydroxyandrostenedione (11bOHA);
17-OH-progesterone (17OHP);
androstenedione (A);
corticosterone (B); and
testosterone (T).

12. The method according to claim 11, wherein the physiological parameters further comprise at least one of the following steroids:
pregnenolone (Preg);
dehydroepiandrosterone (DHEA);
cortisol (F);
cortisone (E);
11-deoxycorticosterone (DOC);
17-OH-pregnenolone (17OHPreg);
aldosterone (ALDO); and
progesterone (P).

13. The method according to claim 11, wherein the physiological parameters comprise steroids only.

14. The method according to am claim 11, wherein the model giving the nonclassical 21-hydroxylase deficiency score (S_{NC2IOHD}) is previously trained by machine learning.

15. The method according to claim 11, wherein the model giving the nonclassical 21-hydroxylase deficiency score comprises a linear combination of the measurements of the physiological parameters.

16. The method according to claim 11, wherein at least one of the steroids was measured during a luteal phase of a menstrual cycle of the female to be assessed.

17. The method according to claim 11, further comprising:
comprising the nonclassical 21-hydroxylase deficiency score (S_{NC2IOHD}) with a predefined threshold, for example 0.5; and
calculating, when the nonclassical 21-hydroxylase deficiency score (S_{NC2IOHD}) is on a predefined side of the threshold, a score (S_{PCOS}) of polycystic ovary syndrome of the female to be assessed, according to a model giving the score (S_{PCOS}) of polycystic ovary syndrome from at least one part of the measurements (p1 . . . p15) received.

18. The method according to claim 17, wherein the polycystic ovary syndrome score (S_{PCOS}) is given from at least the measurements of the steroids: 11β-hydroxyandrostenedione (11bOHA) and androstenedione (A), preferably from measurements of the steroids: testosterone (T) and corticosterone (B).

19. A computer program downloadable from a communication network and/or recorded on a computer-readable medium, comprising instructions for executing a method according to claim 11, when the computer program is executed on a computer.

20. A method for assessing a medical risk of a nonclassical 21-hydroxylase deficiency in a female to be assessed, comprising:
collecting physiological parameters from the female to be assessed, the physiological parameters comprising at least the following steroids: 21-deoxycortisol (21DF), 21-deoxycorticosterone (21DB), 11β-hydroxyandrostenedione (11bOHA), 17-OH-progesterone (17OHP), androstenedione (A), corticosterone (B), and testosterone (T);
calculating a nonclassical 21-hydroxylase deficiency score (S) by introducing these physiological parameters into an automated system according to any one of claims 1 to 6; and
comparing this score (S) with a threshold, the female to be assessed being considered to be at risk of nonclassical 21-hydroxylase deficiency if the score is below the threshold.

21. The method according to claim 20, wherein at least one of the steroids is measured during a luteal phase of a menstrual cycle of the female to be assessed.
